(19) United States
(12) Patent Application Publication
(10) Pub. No.: US 2014/0276557 A1
(43) Pub. Date: Sep. 18, 2014

(54) DUAL RATE INSULIN PUMP

(71) Applicant: Palyon Medical Corporation, Santa Clarita, CA (US)

(72) Inventor: Manish Vaishya, Santa Clarita, CA (US)

(73) Assignee: PALYON MEDICAL CORPORATION, Santa Clarita, CA (US)

(21) Appl. No.: 14/207,924

(22) Filed: Mar. 13, 2014

Related U.S. Application Data
(60) Provisional application No. 61/779,073, filed on Mar. 13, 2013.

(51) Int. Cl. A61M 5/142; A61M 5/168

(52) U.S. Cl. CPC A61M 5/142; A61M 5/16854
USPC 604/505; 604/67

(57) ABSTRACT

A programmable implantable insulin pump is disclosed. The pump includes an implantable pump and a hermetically sealed module. The module provides for varying flow rates of fluid being dispensed from the pump or may provide for a constant flow rate of such fluid. In the case of varying flow rate capabilities, the module preferably includes one or more sensors to determine information relating to the pressure of the fluid, electronics for analyzing the pressure information and determining the flow rate of the fluid, and a mechanism for physically altering the flow rate. First and second resistor capillaries are included in the implantable pump to provide a large range of flow rate capabilities between basal operation and bolus operation. Methods of dispensing a medicament to a patient utilizing such a system are also disclosed, as are variations of the pump system.

### CROSS-REFERENCE TO RELATED APPLICATIONS

The present application claims the benefit of the filing date of U.S. Provisional Patent Application No. 61/779,073 filed Mar. 13, 2013, the disclosure of which is hereby incorporated herein by reference.

### BACKGROUND OF THE INVENTION

The present invention relates to implantable devices, more particularly, programmable implantable pumps allowing for variable flow rates in delivering medication or other fluid to a selected site in the body of a patient.

Implantable pumps have been well known and widely utilized for many years. Typically, pumps of this type are implanted into patients who require the delivery of active substances or medication fluids to specific areas of their body. For example, patients that are experiencing severe pain may require pain killers daily or multiple times per day. Absent the use of an implantable pump or the like, a patient of this type would be subject to one or more painful injections of such medication fluids. In the case of pain associated with more remote areas of the body, such as the spine, these injections may be extremely difficult to administer and particularly painful for the patient. In certain instances, proper application of such medication may be impossible. Furthermore, attempting to treat conditions such as this through oral or intravascular administration of medication often requires higher doses of medication and may cause severe side effects. Therefore, it is widely recognized that utilizing an implantable pump may be beneficial to both a patient and a treating physician.

Implantable pumps have also been used for conditions that require frequent drug delivery. For example, patients suffering from diabetes may have an implantable insulin pump to reduce or eliminate the need for daily insulin injections through the skin. Another key advantage on an implantable insulin pump is optimal dispensing of insulin into peritoneal cavity instead of subcutaneous injection, ease of use by the patient and long refill intervals. Implantable insulin pumps may also reduce problems due to patient compliance, and further may track, store, and/or transmit data relating to treatment for purposes of record keeping and analysis.

Many implantable pump designs have been proposed. For example, commonly invented U.S. Pat. No. 4,969,873 (“the '873 patent”), the disclosure of which is hereby incorporated by reference herein, teaches one such design. The '873 patent is an example of a constant flow pump, which typically includes a housing having two chambers, a first chamber for holding a specific medication fluid to be administered and a second chamber for holding a propellant. A flexible membrane preferably separates the two chambers such that expansion of the propellant in the second chamber pushes the medication fluid out of the first chamber. It is to be understood that the propellant typically expands under normal body temperature. This type of pump also typically includes an outlet opening connected to a catheter for directing the medication fluid to the desired area of the body, a replenishment opening for allowing for refill of the medication fluid into the first chamber and a bolus opening for allowing the direct introduction of a substance through the catheter without introduction into the first chamber. Both the replenishment opening and the bolus opening are typically covered by a septum that allows a needle or similar device to be passed through it, but which properly seals the opening upon removal of the device. As pumps of this type provide a constant flow of medication fluid to the specific area of the body, they must be refilled periodically with the proper concentration of medication fluids suited for extended release.

Although clearly beneficial to patients and doctors that utilize them, constant flow pumps generally have one major problem, i.e., that only a single flow rate can be achieved from the pump. Thus, implantable pumps have also been developed, which allow for variable flow rates of medication therefrom. These pumps are typically referred to as programmable pumps, and have exhibited many different types of designs. For instance, in a solenoid pump, the flow rate of medication fluid can be controlled by changing the stroke rate of the pump. In a peristaltic pump, the flow rate can be controlled by changing the roller velocity of the pump. Likewise, pumps of the constant flow type have been modified to allow for a variable and programmable flow rate. For instance, commonly owned U.S. Pat. No. 7,637,892 (“the '892 patent”) teaches such a design. The '892 patent, as well as related U.S. patent application Ser. Nos. 11/125,586; 11/126,101; 11/157,437; and 13/338,673 are each incorporated herein by reference. In each case, the benefit of providing variable flow is at the forefront, so that differing levels of medication can be delivered to the patient at different times.

In the '892 patent, a constant flow-type pump assembly is modified to include a module that converts the constant flow pump into a programmable pump. That control module includes, inter alia, two pressure sensors, a constant flow capillary, and a valve assembly. The pressure centers are utilized to measure pressure directly from a medication chamber, and pressure just prior to entering the valve assembly. These pressure readings are utilized by a computing unit, which in turn causes a motor to operate the valve assembly to allow lesser or greater flow from the pump. The capillary preferably ensures that a maximum flow rate can only be achieved from the pump. The pump taught in the '892 patent is indeed a useful programmable pump, but one which may be improved.

Certain prior art pumps are used primarily for the delivery of pain medicine. These pumps may be conceptually similar and even structurally similar to pumps to deliver insulin, but improvements may be made to prior art pumps to make them more suitable for the delivery of insulin. For example, a pump for delivering pain medicine may deliver, at a minimum basal rate of approximately 100 μL of medicine per day. A diabetes patient, on the other hand, may require a basal rate of approximately 15 μL of medicine (e.g. insulin) a day. Similarly, pumps for delivery of pain medicine may deliver a maximum flow rate of medicine up to approximately 2 mL per day. Insulin pumps, on the other hand, may be expected to deliver a instantaneous bolus rate of medicine (e.g. insulin) up to approximately 18 mL per day. The ratio between a low basal delivery rate and the maximum bolus delivery rate for pain pumps may thus be about 1:20 (100 μL:2000 μL). The ratio between a low basal delivery rate and the maximum bolus delivery rate for insulin pumps, on the other hand, may be about 1:12,00 (15 μL:18,000 μL). As can be seen, the range of normal and bolus rates for pain pumps and insulin pumps may be quite different. Existing technologies are generally not capable of delivering (a) such low basal rate without severely affecting the flow accuracy and (b) a wide delivery range as foreseeably required for an insulin pump. As such, prior art pumps directed to delivering pain medicine may benefit from modification and/or improvement to better suit the needs of a diabetic patient, particularly in terms of rates of medicine delivery from implantable pumps.

Therefore, there exists a need for an improved programmable implantable pump design.

### BRIEF SUMMARY OF THE INVENTION

A first aspect of the invention is a programmable pump for dispensing a fluid at varying flow rates to a patient. The pump includes a constant flow module including a first chamber housing the fluid, first and second resistor capillaries in fluid communication with the first chamber and a first opening in fluid communication with a catheter. The pump also includes a hermetically sealed control module attached to the constant flow module and including a first motor assembly and valve block, the valve block being in fluid communication with the first and second resistor capillaries and the first opening, the first motor assembly having a first motor and a first valve connected with the motor. The flow rate of the fluid dispelled from the first chamber is affected by varying positioning of the valve. The fluid may be one adapted to treat a diabetic patient, such as insulin.

The first resistor capillary may have a maximum flow rate and the second resistor capillary may also have a maximum flow rate, the maximum flow rate of the first resistor capillary being less than the maximum flow rate of the second resistor capillary. The maximum flow rate of the second resistor capillary may be, for example, at least 200 or 10,000 times greater than the maximum flow rate of the first resistor capillary. On the other hand, the maximum flow rate of the first resistor capillary is designed to be in the vicinity of the minimum flow rate desired of the second resistor capillary.

The pump may include a second valve configured to limit flow of fluid from the second resistor capillary to the valve block. The second resistor capillary may have a first end in fluid communication with the first chamber and a second end in fluid communication with the valve block. The second valve may be positioned after the second end of the second resistor capillary. Alternately, the second valve may be positioned between the first and second end of the resistor capillary.

During operation of the pump, fluid dispelled from the first chamber passes through the first resistor capillary, into the valve block, into contact with the first valve, out of the valve block, into the first opening and through the catheter. The second valve may have an “on” position and an “off” position. When in the “open” position, fluid dispelled from the first chamber passes through the second resistor capillary, into the valve block, into contact with the first valve, out of the valve block, into the first opening and through the catheter. When in the “closed” position, fluid dispelled from the first chamber passes through the second resistor capillary, but does not pass into the valve block. Another embodiment may include one or more intermediate positions of the secondary valve, such as partially open, that allows for additional values of flow as desired.

The constant flow module may further include a second chamber separated from the first chamber by a first flexible membrane. The second chamber may be filled with a propellant that acts upon the flexible membrane to push the fluid from the first chamber through the first and second resistor capillaries. The control module may further include a first pressure sensor for monitoring a pressure of the fluid in the first chamber and a second pressure sensor for monitoring the pressure of the fluid in the valve block.

The pump may further include an enclosure top attached to the constant flow module and covering the control module. The pump may also include a second motor configured to drive the second valve. The pump may also include a motor drive configured to drive the first motor and the second motor.

### BRIEF DESCRIPTION OF THE DRAWINGS

For more complete appreciation of the subject matter of the present invention and the various advantages thereof can be realized by reference to the following detailed description in which reference is made to the accompanying drawings in which:

FIG. 1 is a perspective view of a programmable implantable pump in accordance with one embodiment of the present invention.

FIG. 2 is a top view of the programmable implantable pump shown in FIG. 1.

FIG. 3 is a bottom view of the implantable programmable pump shown in FIG. 1.

FIG. 4 is a right side view of the programmable implantable pump shown in FIG. 1.

FIG. 5 is a left side view of the programmable implantable pump shown in FIG. 1.

FIG. 6 is a rear view of the programmable implantable pump shown in FIG. 1.

FIG. 7 is a front view of the programmable implantable pump shown in FIG. 1.

FIG. 8 is a perspective view of the implantable programmable pump shown in FIG. 1 with an enclosure top removed therefrom.

FIG. 9 is a perspective view of a constant flow module assembly of the programmable implantable pump shown in FIG. 1.

FIG. 10 is a top view of the constant flow module assembly shown in FIG. 9.

FIG. 11 is cross-sectional view of the constant flow module assembly taken along line AA of FIG. 10.

FIG. 12 is a perspective view of a control module assembly of the programmable implantable pump shown in FIG. 1.

FIG. 13 is a top view of the control module assembly shown in FIG. 12.

FIG. 14 is a bottom view of the control module assembly shown in FIG. 12.

FIG. 15 is a perspective view of the control module assembly shown in FIG. 12, with a titanium enclosure top removed therefrom.

FIG. 16 is another perspective view similar to that shown in FIG. 15.

FIG. 17 is a top view of the control module assembly shown in FIGS. 15 and 16.

FIG. 18 is another view of the control module assembly shown in FIGS. 15-17, with an additional portion removed therefrom.

FIG. 19 is a top view of the control module assembly shown in FIG. 18, with a further additional portion removed therefrom.

FIG. 20 is a top view of the control module assembly shown in FIG. 19, with an even further additional portion removed therefrom.

FIG. 21 is a top view of a motor and valve block assembly included in the control module assembly shown in FIG. 12.

FIG. 22 is a top view of a motor, bushing, and valve assembly included in the construct shown in FIG. 21.

FIG. 23 is a top view of the assembly shown in FIG. 22 with the bellows removed therefrom.

FIG. 24 is a view similar to that of FIG. 23, with a stem bushing construct removed therefrom.

FIG. 25 is a top view of the valve block depicted in FIG. 21.

FIG. 26 is a left side view of the valve block shown in FIG. 25.

FIG. 27 is a bottom view of the valve block shown in FIG. 25.

FIG. 28 is a view similar to that shown in FIG. 21, with the valve block shown in phantom.

FIG. 29 is a cross-sectional view taken along line BB of FIG. 26.

FIG. 30 is a perspective view of union nut included in the pump shown in FIG. 1.

FIG. 31 is a top view of an alternate embodiment constant flow module.

FIG. 32 is a top perspective view of the constant flow module shown in FIG. 31.

FIG. 33 is a side cross-sectional view of the constant flow module shown in FIG. 31.

FIG. 34 is a top perspective view of an alternate embodiment control module assembly, with a titanium enclosure top removed therefrom.

FIG. 35 is another top perspective view of the control module assembly shown in FIG. 34, with a titanium enclosure and circuit board removed therefrom.

FIG. 36 is an exploded view of an alternate embodiment motor and valve block assembly included in the control module assembly shown in FIG. 34.

FIG. 37 is another exploded view of the motor and valve block assembly shown in FIG. 34, with certain portions removed therefrom.

FIG. 38 is a schematic view of a medication pump with two resistor capillaries.

FIG. 39 is a sectional view of a dual-resistor capillary pump.

FIG. 40 is a perspective view of the internal components of another embodiment of a dual-resistor capillary pump.

FIG. 41 is a sectional view of a portion of the pump illustrated in FIG. 40.

FIG. 42 illustrates a perspective view of a valve block for a dual-resistor capillary pump.

FIG. 43 illustrates the valve block of FIG. 42 in partial phantom view.

### DETAILED DESCRIPTION

In describing the preferred embodiments of the subject matter illustrated and to be described with respect to the drawings, specific terminology will be used for the sake of clarity. However, the invention is not intended to be limited to any specific terms used herein, and it is to be understood that each specific term includes all technical equivalents which operate in a similar matter to accomplish a similar purpose.

Referring to the drawings, wherein like reference numerals refer to like elements, there is shown in FIGS. 1-7 a programmable implantable pump designated generally by reference numeral 10. As shown in those figures, pump 10 includes a constant flow module assembly 12 (shown alone in FIGS. 9-11), an enclosure top 14, and a union nut 16 (shown alone in FIG. 30). Moreover, as best shown in FIG. 8, where enclosure top 14 has been removed, pump 10 includes a control module assembly 18 engaged with the top portion of constant flow module 12.

In constructing pump 10, control module assembly 18 is placed on top of constant flow module assembly 12, and union nut 16 is threaded onto a threaded portion 20 of the constant flow module (best shown in FIGS. 9-11). A flange 22 formed on control module assembly 18 (best shown in FIGS. 12 and 13) allows for the control module assembly to be captured by the union nut 16 and thusly fixably attached to constant flow module assembly 12. A gasket or the like (shown as element 50 in FIGS. 9 and 10) may be placed between constant flow module 12 and control module assembly 18 so as to ensure a sealed fluid connection between the various corresponding ports of those two components (discussed more fully below). Finally, enclosure top 14 is preferably snapped over the construct to form pump 10 as shown in FIGS. 1-7.

As is also shown in FIGS. 1-7 (as well as other figures), pump 10 also includes suture apertures 24 and a catheter connector 26, both on the constant flow module assembly 12. The former are useful in fixing pump 10 within a patient's body, while the latter is preferably connectable with a longer, and in some cases more flexible, catheter that extends further within the patient's body. Catheter connector 26 preferably includes a strain relief 28 for reducing stresses and strains at or near the connection between catheter 26 and constant flow module assembly 12. Such strain relief can be of any design as are known in the art, and in the embodiment shown, strain relief 28 is designed to slide over catheter 26 and connect with a portion of constant flow module 12.

The constant flow module operates in much of the same fashion as in previous pumps, including those taught in the aforementioned '892 patent, as well as in other commonly owned patents such as U.S. Pat. Nos. 4,969,873, 5,085,656, 5,336,194, 5,836,915, 5,722,957, 5,814,019, 5,766,150 and 6,730,060, the disclosures of which are hereby incorporated by reference herein. Essentially, and as is shown more particularly in the cross-sectional view of FIG. 11, constant flow module assembly 12 includes a medication chamber 30 defined by an upper portion 32 of the constant flow module and a flexible membrane 34, and a propellant chamber 36 defined by membrane 34 and a lower portion 38 of the constant flow module. Like in other pump designs, propellant chamber 36 may in actuality be defined as a propellant pillow consisting of membrane 34 and a lower membrane 34A (not shown). As shown in FIG. 11, propellant chamber 36 is preferably filled utilizing a propellant pillow 37, such as that taught U.S. Pat. No. 5,766,150 or U.S. patent application Ser. No. 12/947,187, the disclosures of which are hereby incorporated by reference herein. As is also shown in FIG. 11, upper portion 32 and lower portion 38 of the constant flow module assembly 12 are preferably screwed together, thereby capturing membrane 34 (and membrane 34A) therebetween. Of course, in other embodiments, other connection means may be employed.

As best shown in FIGS. 9 and 10, constant flow module assembly 12 further includes a catheter access opening 40 through which a portion (e.g., a shoulder shown as a portion of below-discussed gasket 50) 42 of catheter 26 extends, a structure 44, an exit 46, and an entrance/exit 48. More particularly, opening 40 acts to both allow direct injection of fluid through catheter access port and to accept fluid dispelled from control module assembly 18 (as will be discussed more fully below). Structure 44 preferably aids in creating a sealable connection between constant flow module assembly 12 and control module assembly 18 by creating a symmetrical upper surface of assembly 12, thereby evenly spreading compression of a gasket (discussed below) between the two assemblies. Second exit 46 provides fluid to control module assembly 18 to be routed through a valve assembly (also discussed more fully below). Entrance/exit 48 allows for both medication to be injected into chamber 30 and a pressure reading to be taken by a pressure sensor (also discussed more fully below). Assembly 12 also includes a notch 49.

FIGS. 9 and 10 also depict component gasket 50 and circumferential antenna 52. With regard to the former, the gasket is shown as a thin circular portion of silicone or the like which acts to seal around the various openings in flow module assembly 12. Likewise, circumferential antenna 52 is shown as a circular component that fits over threaded portion 20 of the constant flow module and on top of a shoulder formed in the module. This shoulder is better shown in FIG. 11. The antenna is particularly useful in receiving signals emitted from a secondary device during operation or reprogramming of the pump. Circumferential antenna 52 includes a tab 53 which extends into notch 49 so as to be capable of cooperating with control module assembly 18, as will be discussed more fully below. Finally, constant flow module 12 also includes union pins 54a and 54b for engagement with control module 18.

Turning now to FIGS. 12-14, a fully constructed control module assembly 18 is depicted. The module includes two titanium outer portions, namely, upper portion 56 and lower portion 58. Above-discussed flange 22 is formed on lower portion 58. A refill aperture 60 is formed through the center of upper portion 56. A catheter access aperture 62 is formed offset from refill aperture 60. As best shown in FIG. 13, refill aperture 60 allows for a needle to pierce a central septum 64, while catheter access aperture 62 allows for a needle to engage screen member 66. It is to be understood that screen member 66 is designed with a plurality of apertures that are sized so as to prevent needles having a certain size from extending therethrough. This allows for larger needles to be designated for a refill procedure (through central septum 64), while smaller needles are provided for catheter direct access. This is an added safety measure, that is discussed in application Ser. No. 13/276,469 entitled “Mesh Protection System,” and screen member 66 is similar to the like structure formed in that application.

FIG. 14 depicts a view of lower portion 58 of module 18. As shown, lower portion 58 includes several openings, including refill opening 70, reception opening 72, exit opening 74 and electronic access opening 76. An alternate embodiment antenna assembly 77 is shown removed from within electronic access opening 76, but with wires that attach the antenna to the module depicted. It is to be understood that pump 10 can utilize either antenna assembly depicted in the present application, both antenna assemblies, or an alternate assembly not shown herein. Moreover, union pin reception openings 78a and 78b are provided for receiving union pins, 54a and 54b, respectively. Refill opening 70 serves two purposes, namely, allowing for fluid injected through refill aperture 60 to pass into chamber 30 through entrance/exit opening 48, and allowing for access (as will be discussed below) to a pressure sensor disposed within module 18. Reception opening 72 allows for fluid dispelled from exit 46 of constant flow module 12 to be introduced into a valve assembly disposed within module 18. Exit opening 74 overlies opening 40 and shoulder 42 of constant flow module 12 in a fully assembled state. This allows for fluid ultimately dispelled from the valve assembly included within module 18 to flow through catheter 26, and thusly to the patient. Finally, electronic access opening 76 provides a corridor for certain internal electronic structures discussed below to communicate with tab 53 of antenna 52.

FIGS. 15-17 depict module 18 with upper portion 56 removed therefrom. As shown, within its interior, module 18 includes a circuit board 80, a first pressure sensor 82, a second pressure sensor 84, a valve block 86, a motor assembly 88, a buzzer 90, and a flexible conductive element 92. FIGS. 16 and 17 depict similar views to FIG. 15, albeit from different perspectives. Circuit board 80 is held to a circuit board support 94, which is better shown in FIG. 18 where circuit board 80 is removed. Screws 96a-96d hold circuit board 80 to circuit board support 94. Flexible conductive element 92 preferably provides electrical interconnection among circuit board 80, first pressure sensor 82, second pressure sensor 84, motor assembly 88 and buzzer 90. Module further includes a feed through 98, which is also preferably connected with flexible conductive element 92, and which extends through electronic access opening 76 on the bottom of module 18. This element preferably provides the interconnection of the internals of module 18 with antenna 52, specifically tab 53.

As noted above, FIG. 18 depicts the internals of module 18 with circuit board 80 removed therefrom. In this view, it is shown that module 18 also includes batteries 100a and 100b for powering the pump. Also shown, is the interconnection among flexible conductive element and flexible conductive element 92 and both batteries. FIG. 19 shows the internal structure of module 18, this time with circuit board support 94 removed therefrom. In this figure, the configuration and interconnection among the elements and flexible conductive element 92 are further depicted. In the embodiments shown, flexible conductive element is constructed of a polymide material, but can be any other conductive element, including wires or the like. Also more clearly shown in FIGS. 18 and 19 is the connection between motor assembly 88 and lower portion 58. Specifically, a set screw 102 is provided at one end of the motor assembly and threaded into a portion of lower portion 58. Moreover, FIG. 19 shows apertures 104a-d, which are designed to accept screws 96a-96d, respectively. Thus, circuit board is held tightly not only to circuit board support 94, but also lower portion 58.

FIG. 20 depicts module 18 in a similar view to that of FIG. 19, but with flexible conductive element 92 and batteries 100a and 100b being removed therefrom. In this view, a capacitor 106 is shown. This component allows for the generation of higher voltage than batteries 100a and 100b themselves. In general, capacitor 106 operates like a standard capacitor, storing charge for use in powering the pump. It is to be understood that capacitor 106 could be removed depending upon the particular batteries that are utilized. For instance, batteries that generate higher voltages and less current typically will negate the need for a capacitor. However, batteries suitable for inclusion in module 18 tend to be produced in the lower voltage range (3.2V-3.8V). Moreover, smaller capacitors could be included on circuit board 80 to achieve the same goal as capacitor 106.

FIGS. 21 and 25-29 focus on valve block 86, its internal components, and its cooperation with motor assembly 88. As shown, valve block 86 includes a pressure sensor receiving aperture 106, as well as catheter access aperture 62. Pressure sensor receiving aperture 106 is designed to receive second pressure sensor 84, as well as allow for fluid to come into contact with that pressure sensor. Valve block 86 also includes a first body portion 108 and a second body portion 110. First body portion 108 includes apertures 62 and 106, as well as several fluid passageways and a valve receiving channel (best shown in FIG. 28) for allowing fluid flow within the valve block and ultimately to the patient. Second body portion 110 is essentially a hollow cylindrical body, the interior of which is designed to receive a portion of the valve. This again is best shown in FIG. 28, with FIG. 26 depicting a front view of same. It is noted that valve block 86 is depicted by itself in FIGS. 25-27, with FIG. 27 depicting a bottom surface thereof. As shown in that drawing, apertures 62a and 106a cooperate with the above discussed apertures 62 and 106, respectively.

As also shown in FIG. 21, motor assembly 88 is connected with valve block 86 by two screws 112a and 112b, which extend through apertures in a flange portion 114 of the motor assembly, and into apertures 116a and 116b, respectively, of the valve block (best shown in FIG. 26). This cooperation fixably connects motor assembly 88 with valve body 86. As noted above, motor assembly 88 is also connected to module 18 via set screw 102. Likewise, valve block 86 is connected to other portions of module 18 via pin 118, as best shown in FIG. 26. That pin preferably includes a bulbous head portion that, once inserted within a hole in module 18, acts to prevent removal of the valve block.

FIG. 22 depicts motor assembly 88 without valve body 86, and highlights the portions of the assembly that extend into the valve body. Specifically, motor assembly 88 includes a bellows 120, valve 122, and an o-ring 124. Bellows 120 is preferably welded to weld ring 126, which in turn is welded to flange 114. Likewise, bellows 120 is preferably welded to valve at surface 128. Referring now to FIG. 23, in which bellows 120 is removed, it is shown that valve 122 consists of a valve stem 130 which extends through a valve bushing 132. It is around this valve bushing that o-ring 124 is disposed. Valve stem 130 includes at a distal end a tapered portion. FIG. 24 on the other hand depicts the assembly with a motor housing 134 removed therefrom. In this view, weld ring 126 is clearly shown. Also shown is a motor mount plug 136 which screwably connects with motor housing 134.

Motor 89 of motor assembly 88 is preferably a piezoelectric motor, as such a motor does not include a permanent magnet, which makes the motor MRI compatible. In addition, piezoelectric motors are generally of a smaller size and require less energy for operation. Still further, piezoelectric motors operate in a straight line, which is ideal in the present instance, as will be discussed below. However, it is to be understood that motor 89 could be other types of motors, including stepper motors or the like. Of course, certain of the above-mentioned benefits of the piezoelectric motor may not be met by such alternate motor designs. Operation of motor 89 imparts a force upon valve stem 130, which moves within second body portion 110 of valve block 86. The combination of bellows 120 and o-ring 124 insures that any fluid flowing within valve block 186 cannot seep outside of that component. In other words, bellows 120 and o-ring 124 insure a sealable connection between motor assembly 88 and valve block 86. As is shown in FIGS. 28 and 29, the most distal portion of valve stem 130 extends within the fluid flow path, and the conical nature of that distal portion provides that movement of the valve stem results in greater or lesser fluid flow threw valve block 86. The inclusion of a stepper motor such as the one discussed above insures that fine adjustments of flow rate through the valve block can be realized. In fact, movement of the valve relates in a linear or near linear fashion to the flow rate. The above-discussed sealable nature of bellows 120 and o-ring 124 insures hermetic sealing within the valve block, and thusly prevents fluid from flowing anywhere other than the valve block. This is particularly important given the other components of module 18.

In the embodiment shown, valve stem 130 and valve portion 132 are shown as constructed of titanium material. It is to be understood that any suitable material may be employed. Moreover, it is to be understand that valve stem 130, at its most distal end, could include a silicon covering or the like in order to insure a full closure of the valve if desired. Likewise, while o-ring 124 as shown as being constructed of a silicon material, any other suitable material may be employed. For instance, Teflon may be employed, as can a material known as PORON®.

In operation, fluid dispelled from chamber 30 (under pressure provided by chamber 36) travels through both exits 46 and 48. The fluid dispelled through exit 48 is preferably directed into contact with first pressure sensor 82, so a pressure reading of the fluid within chamber 30 can be taken. The fluid dispelled through exit 46 preferably first travels through a filter and capillary construction, as are known in the art. In one example of such a structure, a filter and capillary are coiled around an underside of upper portion 32. Fluid flows through the filter, which is designed to prevent particulates and other undesirable matter of flowing into the capillary, and thereafter flows through the capillary, which is essentially a very small tube with a small diameter that allows a maximum flow rate of fluid therethrough. That fluid then flows through aperture 106a and into the passages provided in valve block 86. Second pressure sensor 84 takes a pressure reading of the fluid within the valve block.

Once within valve block 86, the fluid flows into contact with the distal end of valve stem 130. Depending upon the positioning of the valve stem, the flow of the fluid will either be reduced or remain the same as the maximum flow rate dictated by the aforementioned capillary. Second pressure sensor 84 is positioned to take a reading of the pressure before the valve portion, and thusly the comparison of the readings taken by first pressure sensor 82 and second pressure sensor 84 can be utilized to determine the actual flow rate of the fluid after passing through the resistor and the valve. This is preferably determined by circuit board 80, as sensors 82 and 84 are electrically connected thereto by flexible conductive element 92. If the flow rate is not desired, motor 89 can be operated to vary the position of valve stem 130. Subsequent to contacting the valve, fluid flows through other passages formed in valve block 86, through aperture 62a and ultimately through catheter 26. Depending upon the placement of the catheter within the patient, the fluid is delivered to the desired portion of the patient in which the catheter is directed.

It is to be understood that pump 10 preferably operates with little outside interaction required. Aside from refilling chamber 30 with an active substance, a doctor or other medical professional likely only needs to interact with the pump in order to set a desired flow rate. This may be accomplished through the use of a wand or other transmitter/receiver (not shown) that interfaces with antenna 92. Once the flow rate is set, pump 10 preferably operates on its own to maintain the flow rate. Pump 10 may also be programmed to provide different flow rates at different times of the day. For instance, patients may require lesser doses of pain medication while sleeping, and heavier doses of pain medication upon waking up. Similarly, diabetic patients may need higher doses of insulin prior to eating a meal or lower doses of insulin during heavy exercise. Circuit board 80 can be designed to allow for such programming. Above-noted buzzer is designed to emit an audible warning upon certain conditions, including low battery, low fluid level within chamber 30, low or high temperature conditions, and high pressure, which may indicate overfilling of chamber 30, low pressure differential across the resistor capillary or blockage within catheter 26. Upon recognizing the audible sound, the patient can contact his or her medical professional.

Valve 122 may also include a positioning sensor (not shown) or the like associated therewith. Such a sensor may be capable of providing information relating to the positioning of the valve to circuit board 80. Such positioning sensors can include many different designs. For example, light reflective technology can be employed to determine at any given moment the position of the valve. Likewise, valve 122 may be provided with one or more conductive elements that interact with conductive elements provided on or near valve block 86. The completion of an electrical circuit in such a case can indicate the positioning of valve 122. Still further, the positioning sensor can take the form of an induction coil capable of determining the positioning of the valve therein. A slide potentiometer may also be employed, as can a stack switch.

During a refill procedure, pump 10 can be monitored through the use of the wand or other transmitter/receiver. A computer program associated with such device and pump 10 can indicate to the doctor whether the refill needle is correctly placed within the pump. Known problems with refilling implantable pumps are misapplications of a refill needle to the tissue of the patient (so called pocket fills) and to a bolus opening such as catheter access aperture 62. Directly injecting a patient with a dose of medication meant for prolonged release from chamber 30 can have dire consequences. During the monitoring of the refill procedure, a quick change in pressure within chamber 30 can be recognized by the medical professional, thereby ensuring placement of the needle within refill aperture 60. This is a significant safety feature in pump 10.

The exterior portions of pump 10 are preferably constructed of PEEK, including constant flow module assembly 12, enclosure top 14 and union nut 16. On the other hand, the exterior portions of control module assembly are constructed of titanium, which ensures the hermetic nature of that component. However, certain interior portions of the module are also constructed of PEEK, including circuit board support 94. While these are indeed the materials utilized in the construction of a preferred pump 10, other materials may be employed in other embodiments. For instance, other polymeric materials may be employed that provide for similar strength, while maintaining the low overall weight provided for by the PEEK material. Likewise, other metallic materials may be substituted for titanium, such as stainless steel or the like. The only limitation is that the materials selected should be bio-compatible to ensure such are not rejected by the patient after implantation.

Several variations of above-discussed pump 10 will now be discussed. It is to be understood that all or some of these variations may be incorporated into an implantable pump according to the present invention. Where possible, like elements to those discussed above are referred with reference numerals in a different 100-series of numbers.

For instance, FIG. 31 depicts a top portion of an alternate embodiment constant flow module 312, which includes a differently shaped gasket 350. That gasket has been removed from FIG. 32. In this embodiment, a portion 342 stands alone as part of catheter 326. FIG. 33 depicts a side cross section of constant flow module 312. As is seen in this view, module 312 differs from that of module 12 in that a bottom thereof is no longer contoured, but rather, exhibits a flat configuration. Constant flow module 312 has also been provided with two o-rings 313a and 313b. Where ring 313a ensures a sealing of the propellant and medication chambers of module 312, ring 313b ensures no material can leak from module 312. Still further, module 312 includes holes 315a-c. Hole 315a preferably receives a pin or the like (not shown) that acts to prevent the two housing portions included in module 312 from inadvertently disengaging by preventing unscrewing of those portions. On the other hand, holes 315b and 315c aid in connecting those portions to each other. Specifically, holes 315b and 315c are capable of interfacing with a tool for use in screwing the module portions together. Of course, other embodiments may include any number of similar holes.

FIG. 34 depicts an alternate embodiment control module assembly 318 in which an element similar to the above-discussed flexible conductive element 92 has been eliminated. In this embodiment assembly 318, a circuit board 380 acts to connect all of the electrical elements of the module. FIG. 35 depicts the module 318 with circuit board 380 removed.

FIGS. 36 and 37 depict alternate embodiment valve block 386 and motor assembly 388, as well as the cooperation of those two elements. The major differences between this embodiment and those discussed above lies in several areas. For one, valve 422 includes a valve stem 430, which includes an overmolded silicone valve tip 432. This tip ensures full seating within a valve seat (not shown) located in block 386, as well as allows for fine adjustment of flow rates therethrough. In addition, motor assembly 388 includes a solid housing 434, and does not include a portion similar to plug 136. Finally, motor 389 is held in place by clamp elements 389a and 389b. Both elements are fitted into or onto different portions of the motor and thereafter affixed to block 386, preferably through the use of epoxy.

FIG. 38 illustrates a schematic view of an alternate embodiment of a pump 510. Pump 510 is structurally similar to other embodiments described above in many ways, but may include features particularly adapted for use in implantable insulin pumps. For example, the pump 510 may include a first resistor capillary 511 and a second resistor capillary 513. The first resistor capillary 511 may be, for example, a low flow resistor capillary. This first resistor capillary 511 may be smaller than one used for a pump with pain medicine, as the basal delivery rate for insulin may be significantly less than the basal delivery rate for pain medicine in patients. However, to be able to reach the higher delivery rates of insulin that may be required during bolus delivery, a second resistor capillary 513 may be a high flow resistor capillary, larger than the first resistor capillary 511 and larger than resistor capillaries that may be used in pumps that deliver pain medicine.

Both the first and second resistor capillaries 511, 513 are in fluid communication with a medication chamber 530, which may include one of various drugs. Preferably, the medication chamber 530 contains a drug useful in the treatment of diabetes, such as insulin or an insulin analog or derivative. As in embodiments described above, a first pressure sensor 582 is located in the pump housing in fluid communication with medication chamber 530 and is configured to take a pressure reading of the fluid in the medication chamber. A first end of the first resistor capillary 511 is in fluid communication with the medication chamber 530, and a second end of the first resistor capillary is in fluid communication with a second pressure sensor 584. Similarly, a first end of the second resistor capillary 513 is in fluid communication with the medication chamber 530, and a second end of the second resistor capillary 513 is in fluid communication with the second pressure sensor 584. The second pressure sensor 584 is similar to that described in embodiments above, and is configured to take a second pressure reading of the medication fluid upon exiting one or both of the resistor capillaries.

A shut-off valve 515 may be interposed between the first and second ends of the second resistor capillary 513. The shut-off valve 515 may alternately be positioned after the second end of the second resistor capillary 513. The shut-off valve 515 may be configured to allow a user to selectively interrupt the fluid communication between the second resistor capillary 513 and the second pressure sensor 584, as well as the remainder of an outflow portion of the pump 510. The shut-off valve 515 may be operably connected to electronics within the pump 510, for example a motor drive 517, that communicates with the shut-off valve, causing the shut-off valve to alternate from an open position to a closed position, or vice versa. In one embodiment, the motor drive 517 that operates the shut-off valve 515 also operates the valve block 586 in a similar fashion as described above. The motor drive 517 may alternately communicate between the shut-off valve 515 or the valve block 586, for example, depending on the status of a switch 519.

In operation, the pump 510 works much the same as in embodiments described above. Insulin or other fluid dispelled from the medication chamber 530 (under pressure provided by a propellant chamber) travels through a filter capillary, as is known in the art, and into a first resistor capillary 511 and a second resistor capillary 513. The fluid is also forced into contact with a first pressure sensor 582, which may be effected in the manner described above in relation to other embodiments of the pump. This allows a pressure reading of the medication chamber 530 to be taken. If the shut-off valve 515 is in the closed position, fluid in the second resistor capillary 513 does not pass the shut-off valve 515. Fluid in the first resistor capillary 511 flows through an aperture, as described above in other embodiments of the pump, and into passages provided in valve block 586. The second pressure sensor 584 takes a pressure reading of the fluid within the valve block 586. The fluid continues traveling through the pump 510 and in to the patient in the same or a similar manner as described above with relation to other embodiments of the pump.

When the shut-off valve 515 is in the closed position, the maximum flow rate is limited by the maximum flow rate of the smaller first resistor capillary 511. The flow rate may be further decreased, as described above, using valve block 586. If the shut-off valve 515 is in the open position, the second resistor capillary 513 is in fluid communication with the remainder of the pump 510 and fluid travels through the second resistor capillary 513, feeding into the valve block 586 and eventually the patient. Generally, it is contemplated that the shut-off valve 515 would be switched to the closed position during delivery of a medicine at a basal rate, while the shut-off valve would be switched to the open position during delivery of medicine at a bolus rate that is greater than the basal rate. The higher bolus rate may be useful, for example, just prior to a diabetic patient eating a meal.

In cases in which the shut-off valve 515 is open and in which the second resistor capillary 513 is much larger (in inner diameter) than the first resistor capillary 511, the maximum flow rate of the fluid into the patient is essentially the maximum flow rate allowed by the second resistor capillary 513. Even though fluid is flowing through both resistor capillaries 511, 513, the second resistor capillary will often be so much larger than the first resistor capillary that the additional flow rate provided by the first resistor capillary is negligible compared to the maximum flow rate of the second resistor capillary.

An additional shut-off valve (not illustrated) may be provided between the first and second ends of the first resistor capillary 511, such that the first resistor capillary could be blocked when the second resistor capillary 513 is opened. This may help ensure very precise maximum flow rates if desired, but may be generally unnecessary when the maximum flow rate of the second resistor capillary 513 is much larger than the maximum flow rate of the first resistor capillary.

As described in other embodiments of the pump, the pressure readings taken from the first and second pressure sensors 582, 584 may provide information about flow rate to decide whether, and to what degree, the flow rate should be slowed by changing the position of the valve block 586. In the proposed method with two resistor capillaries, no additional pressure sensors are required in addition to the prior art with single resistor capillary. This is achieved through software wherein based on the position of shutoff valve, the equations for computing flow rates are adjusted accordingly using the same two pressure sensors. Also as in other embodiments described herein, a catheter access aperture 562 may be provided to allow direct injection of a fluid into the catheter 526, bypassing the majority of the pump 510.

FIG. 39 illustrates a cross section of one embodiment of the pump 510. The pump is similar to those described above, with a vertical shut-off valve 515. The shut-off valve 515 is controlled by an actuator, such as a membrane piezoelectric actuator 516. As described above, when the actuator 516 is in a first position, the shut-off valve 515 allows the medication fluid through the second resistor capillary 513 and into the valve block 586. When the actuator 516 is in a second position, the shut-off valve 515 blocks the medication fluid from flowing through the second resistor capillary 513 and into the valve block 586. FIGS. 40-41 illustrate another embodiment of the pump 510 with an alternate shut-off valve 515′ and actuator 516′. In this embodiment, the shut-off valve 515′ is driven by a linear piezoelectric actuator 516′ to block or allow flow from the second resistor capillary 513.

An embodiment of the valve block 586 is illustrated in FIGS. 42-43. FIG. 43 particularly illustrates one embodiment of fluid flow pathways. Fluid flowing through the second resistor capillary 513 flows into the valve block 586 through high-flow inlet 600. When the shut-off valve 515 is open, the fluid continues through the high-flow outlet 602 into the valve block 586. Similarly, fluid flowing through the first resistor capillary 511 enters the valve block 586 through low-flow inlet 604. The fluid entering through the high-flow outlet 602 and the low-flow inlet 602 enters into a chamber where the fluid may mix. From that chamber, the combined fluid continues through the valve block 586 through combined inlet 606, where the fluid flow may be further restricted in essentially the same fashion as described above with reference to valve block 86. The fluid continues through the valve block 586 and exits the pump 510 through delivery outlet 608. This fluid is delivered in essentially the same fashion as described above with relation to the pain medication exiting the pump through a delivery catheter.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A programmable pump for dispensing a fluid at varying flow rates to a patient comprising:
a constant flow module including a first chamber housing the fluid, first and second resistor capillaries in fluid communication with the first chamber, and a first opening in fluid communication with a catheter;
a control module attached to the constant flow module and including a first motor assembly and valve block, the valve block being in fluid communication with the first and second resistor capillaries and the first opening, the first motor assembly having a first motor and a first valve connected with the first motor;
a second valve configured to limit flow of fluid from the second resistor capillary to the valve block;
a first pressure sensor configured to determine a first pressure of the fluid within the first chamber; and
a second pressure sensor configured to determine a second pressure of the fluid within the valve block,
wherein the flow rate of the fluid dispelled from the first chamber is affected by varying positioning of the valve.

2. The programmable pump of claim 1, wherein the fluid is a medicine adapted to treat a diabetic patient.

3. The programmable pump of claim 2, wherein the fluid is insulin, an insulin analog, or an insulin derivative.

4. The programmable pump of claim 1, wherein the first resistor capillary has a first maximum flow rate and the second resistor capillary has a second maximum flow rate, the first maximum flow rate being less than the second maximum flow rate.

5. The programmable pump of claim 4, wherein the second maximum flow rate is between approximately 50 and approximately 1,000 times greater than the first maximum flow rate.

6. The programmable pump of claim 4, wherein the second resistor capillary has a first end in fluid communication with the first chamber and a second end in fluid communication with the valve block.

7. The programmable pump of claim 6, wherein the second valve is positioned downstream of the second end of the second resistor capillary.

8. The programmable pump of claim 6, wherein the second valve is positioned between the first and second ends of the second resistor capillary.

9. The programmable pump of claim 6, wherein during operation of the pump, fluid dispelled from the first chamber passes through the first resistor capillary, into the valve block, into contact with the first valve, out of the valve block, and through the catheter.

10. The programmable pump of claim 9, wherein the second valve has an open position and a closed position.

11. The programmable pump of claim 10, wherein during operation of the pump and when the second valve is in the open position, fluid dispelled from the first chamber passes through the second resistor capillary, into the valve block, into contact with the first valve, out of the valve block, and through the catheter.

12. The programmable pump of claim 11, wherein during operation of the pump and when the second valve is in the closed position, fluid dispelled from the first chamber passes through the first end of the second resistor capillary but not beyond the second end of the resistor capillary.

13. The programmable pump of claim 4, further comprising a second motor configured to drive the second valve.

14. The programmable pump of claim 13, further comprising a first motor drive configured to drive the first motor.

15. The programmable pump of claim 14, wherein the first motor drive is configured to drive the second motor.

16. The programmable pump of claim 14, wherein a second motor drive is configured to drive the second motor.

17. The programmable pump of claim 1, wherein the pump employs exactly two pressure sensors to measure the flow rate of the fluid.

18. The programmable pump of claim 13, wherein the second motor is a linear piezoelectric actuator.

19. The programmable pump of claim 13, wherein the second motor is a piezoelectric membrane.

20. The programmable pump of claim 4, wherein the second maximum flow rate is approximately 200 times greater than the first maximum flow rate.

21. The programmable pump of claim 10, wherein the second valve has a plurality of discrete positions between the open position and the closed position.

22. The programmable pump of claim 14, wherein the motor drive is configured to drive the first and the second motor non-simultaneously.

23. A programmable pump for dispensing a fluid at varying flow rates to a patient comprising:
a constant flow module including a first chamber housing the fluid and a first opening in fluid communication with a catheter;
a first resistor capillary in fluid communication with the first chamber and having a first maximum flow rate;
a second resistor capillary in fluid communication with the first chamber and having a second maximum flow rate between approximately 50 and approximately 1,000 times greater than the first maximum flow rate;
a control module attached to the constant flow module and including a first motor assembly and valve block, the valve block being in fluid communication with the first and second resistor capillaries and the first opening, the first motor assembly having a first motor and a first valve connected with the first motor; and
a second valve configured to limit flow of fluid from the second resistor capillary to the valve block;
a first pressure sensor configured to determine a first pressure of the fluid within the first chamber; and
a second pressure sensor configured to determine a second pressure of the fluid within the valve block,
wherein the flow rate of the fluid dispelled from the first chamber is affected by varying positioning of the valve and the pump employs exactly two pressure sensors to measure the flow rate of the fluid.

24. A method of dispensing a fluid at varying flow rates from a programmable pump to a patient comprising:
simultaneously dispensing the fluid from a first chamber through a first resistor capillary having a first maximum flow and through a second resistor capillary having a second maximum flow rate between approximately 50 and approximately 1,000 times greater than the first maximum flow rate;
operating a first motor to change a position of a first valve to vary a flow rate of the fluid passing from the first and second resistors through a valve block that houses the first valve;
operating a second valve to limit flow of the fluid from the second resistor capillary to the valve block;
determining a first pressure of the fluid within the first chamber with a first pressure sensor; and
determining a second pressure of the fluid within the valve block with a second pressure sensor.
